(19) United States
(12) Patent Application Publication
(10) Pub. No.: US 2023/0263630 A1
(43) Pub. Date: Aug. 24, 2023

(54) DELIVERY APPARATUS FOR PROSTHETIC IMPLANTS

(71) Applicant: EDWARDS LIFESCIENCES CORPORATION, Irvine, CA (US)

(72) Inventors: Tomer Saar, Pardes Hanna-Karkur (IL); Eran Goldberg, Nesher (IL)

(21) Appl. No.: 18/141,800

(22) Filed: May 1, 2023

Related U.S. Application Data
(63) Continuation of application No. PCT/US2021/057703, filed on Nov. 2, 2021.
(60) Provisional application No. 63/108,520, filed on Nov. 2, 2020.

(51) Int. Cl. A61F 2/24

(52) U.S. Cl. CPC A61F 2/2436

(57) ABSTRACT

A delivery apparatus for an expandable prosthetic heart valve includes a handle, and a shaft having a proximal end portion coupled to the handle and a distal end portion including a delivery capsule. The delivery capsule having an inner surface, an outer surface, and a plurality of elongate and axially extending inner and outer guide rails, the inner guide rails being spaced apart circumferentially around and extending radially inwardly from the inner surface and the outer guide rails being spaced apart circumferentially around and extending radially outwardly from the outer surface. The inner guide rails are configured to engage the prosthetic heart valve in a radially-compressed configuration and the outer guide rails are configured to contact an inner surface of an outer shaft.

### CROSS REFERENCE TO RELATED APPLICATIONS

This application is a continuation of International Patent Application No. PCT/US2021/057703, filed Nov. 2, 2021, which claims the benefit of U.S. Provisional Application No. 63/108,520, filed Nov. 2, 2020. The prior applications are incorporated herein by reference in their entirety.

### FIELD

The present disclosure relates to delivery apparatus and methods for implanting prosthetic devices, such as prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic heart valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Initially, the prosthetic heart valve is disposed in a radially-compressed configuration within a capsule of the delivery apparatus. In the radially-compressed configuration, the prosthetic heart valve is inserted into and advanced through the vasculature of a patient to an implantation location (e.g., a native heart valve region). The prosthetic heart valve is deployed from the capsule and expanded from the radially-compressed configuration to the a radially-expanded, functional configuration.

Despite widespread use, typical delivery apparatus and/or methods of implanting prosthetic heart valves have their shortcomings. As such, there is a need for improved delivery apparatus and implantation methods.

### SUMMARY

Described herein are prosthetic valve delivery assemblies and related methods, which can be used to deliver a prosthetic valve to a location within a body of a subject. In some implementations, the prosthetic valve delivery assemblies can be used to deliver a medical device through the vasculature, such as to a heart of the subject.

In one representative example, a delivery apparatus for an expandable prosthetic heart valve is provided. The delivery apparatus includes a handle, and a shaft having a proximal end portion coupled to the handle and a distal end portion including a delivery capsule. The delivery capsule has an inner surface, an outer surface, and a plurality of elongate and axially extending inner and outer guide rails. The inner guide rails being spaced apart circumferentially around and extending radially inwardly from the inner surface and the outer guide rails being spaced apart circumferentially around and extending radially outwardly from the outer surface. The inner guide rails are configured to engage the prosthetic heart valve in a radially-compressed configuration and the outer guide rails are configured to contact an inner surface of an outer shaft.

In another representative example, a delivery apparatus for an expandable prosthetic heart valve includes a handle, and a first shaft and a second shaft extending over the first shaft, each shaft having a distal end portion and a proximal end portion coupled to the handle, the distal end portion of the second shaft having a delivery capsule including an inner surface, an outer surface, and a plurality of elongate positioning ribs extending axially along and circumferentially arranged around the inner surface and the outer surface. The positioning ribs of the inner surface are configured to capture the prosthetic heart valve in a radially-compressed configuration and the positioning ribs of the outer surface are configured to contact an outer shaft through which the delivery capsule can be inserted.

In one representative example, a method for delivering a prosthetic heart valve within a native annulus of a patient is provided. The method includes advancing into a native vasculature of a patient a prosthetic heart valve mounted in a radially-compressed configuration around a distal end portion of a first shaft and engaged by a plurality of elongate guide rails spaced apart circumferentially within a delivery capsule of a second shaft extending over the distal end portion of the first shaft, wherein one or more outwardly extending protrusions of the prosthetic heart valve are received in a recess of the guide rails such that the prosthetic heart valve rotates with rotation of the delivery capsule, inserting the delivery capsule and the prosthetic heart valve into a native annulus of the patient, rotating the delivery capsule and prosthetic heart valve relative to the native vasculature and native annulus of the patient such that the prosthetic heart valve is oriented for implantation into the native annulus, retracting the second shaft such that the prosthetic heart valve and one or more outwardly extending protrusions move axially along the guide rails of the delivery capsule as the delivery capsule is withdrawn, and expanding the prosthetic heart valve from a radially-compressed configuration to a radially-expanded state within the native annulus.

In another representative example, a method for positioning a prosthetic heart valve for implantation into an annulus of a patient includes positioning a delivery capsule extending over a prosthetic heart valve in a radially-compressed configuration and mounted around a distal end portion of a shaft within an annulus of a patient, wherein the prosthetic heart valve is held rotationally stationary relative to the delivery capsule by a plurality of elongate and axially extending positioning ribs which mate with one or more outwardly extending protrusions of the prosthetic heart valve; and rotating and orienting via the delivery capsule and the positioning ribs thereof the prosthetic heart valve within the annulus of the patient such that one or more proximate native lumen are unobstructed upon implantation.

In another representative example, an expandable prosthetic heart valve delivery assembly is provided. The delivery assembly includes a delivery apparatus. The delivery apparatus includes a handle, a first shaft, and a second shaft extending over the first shaft, each shaft having a proximal end portion coupled to the handle and a distal end portion. The distal end portion of the second shaft includes a delivery capsule having an inner surface, an outer surface, and a plurality of elongate and axially extending inner and outer guide rails, each of the inner guide rails having a recess, and an expandable prosthetic heart valve mounted in a radially-compressed configuration around the distal end portion of the first shaft and retained within the delivery capsule of the second shaft. The recesses of the inner guide rails of delivery capsule mate with one or more outwardly extending protrusions of the prosthetic heart valve such that the prosthetic heart valve is rotationally stationary relative to the delivery capsule such that prosthetic heart valve rotates with rotation of the delivery capsule.

In one representative example, a delivery capsule for a prosthetic heart valve delivery apparatus includes a main body having an inner surface and an outer surface, a plurality of inner elongate ribs extending axially along the inner surface of the main body, and a plurality of outer elongate ribs extending axially along the outer surface of the main body. The inner ribs of the main body are configured to engage and hold a prosthetic heart valve rotationally stationary relative to the main body while the prosthetic heart valve is in a radially-compressed configuration.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of an exemplary delivery assembly comprising a mechanically-expandable prosthetic heart valve and a delivery apparatus.

FIG. 2 is a perspective view of the prosthetic heart valve of FIG. 1.

FIG. 3 is a side view of a frame of the prosthetic heart valve of FIG. 1 in a radially-compressed configuration.

FIG. 4 is a perspective view of the frame and actuators the prosthetic heart valve of FIG. 1, depicting the frame in a radially-expanded configuration.

FIG. 5 is a perspective view of a delivery capsule of the delivery apparatus, according to one example.

FIG. 6 is an end view of the delivery capsule of FIG. 5.

FIG. 7 is a partial cross-sectional view of an example of an inner guide rail of the delivery capsule of FIG. 5, taken in a plane perpendicular to the longitudinal axis of the delivery capsule.

FIG. 8 is a partial cross-sectional view of another example of an inner guide rail of the delivery capsule of FIG. 5, taken in a plane perpendicular to the longitudinal axis of the delivery capsule.

FIG. 9 is a partial cross-sectional view of another example of an inner guide rail of the delivery capsule of FIG. 5, taken in a plane perpendicular to the longitudinal axis of the delivery capsule.

FIG. 10 is a partial cross-sectional view of another example of an inner guide rail of the delivery capsule of FIG. 5, taken in a plane perpendicular to the longitudinal axis of the delivery capsule.

FIG. 11 is a partial cross-sectional view of an example of an inner guide rail of the delivery capsule of FIG. 5, taken along a longitudinal axis of the inner guide rail.

FIG. 12 is a partial cross-sectional view of another example of an inner guide rail of the delivery capsule of FIG. 5, taken along a longitudinal axis of the inner guide rail.

FIG. 13 is a partial cross-sectional view of another example of an inner guide rail of the delivery capsule of FIG. 5, taken along a longitudinal axis of the inner guide rail.

FIG. 14 is an end view of a delivery capsule, according to another example.

FIG. 15 is an end view of a delivery capsule, according to yet another example.

FIG. 16 is an end view of the delivery capsule of FIG. 15 together with an introducer and a prosthetic heart valve, which are shown schematically.

FIG. 17 is a partial cross-sectional view of an example of an inner guide rail of the delivery capsule of FIG. 15, taken in a plane perpendicular to the longitudinal axis of the delivery capsule.

FIG. 18 is a partial cross-sectional view of another example of an inner guide rail of the delivery capsule of FIG. 15, taken in a plane perpendicular to the longitudinal axis of the delivery capsule.

FIG. 19 is a partial cross-sectional view of another example of an inner guide rail of the delivery capsule of FIG. 15, taken in a plane perpendicular to the longitudinal axis of the delivery capsule.

FIG. 20 is a partial cross-sectional view of another example of an inner guide rail of the delivery capsule of FIG. 15, taken in a plane perpendicular to the longitudinal axis of the delivery capsule.

FIG. 21 is a partial cross-sectional view of another example of an inner guide rail of the delivery capsule of FIG. 15, taken in a plane perpendicular to the longitudinal axis of the delivery capsule.

FIG. 22 is a partial cross-sectional view of another example of an inner guide rail of the delivery capsule of FIG. 15, taken in a plane perpendicular to the longitudinal axis of the delivery capsule.

FIG. 23 is a partial cross-sectional view of another example of an inner guide rail of the delivery capsule of FIG. 15, taken in a plane perpendicular to the longitudinal axis of the delivery capsule.

FIG. 24 is a partial cross-sectional view of another example of an inner guide rail of the delivery capsule of FIG. 15, taken in a plane perpendicular to the longitudinal axis of the delivery capsule.

FIG. 25 is a perspective view of a distal end portion of a delivery assembly comprising the prosthetic heart valve of FIG. 1 and the delivery capsule of FIG. 15, depicting the prosthetic heart valve partially disposed within the delivery capsule.

FIG. 26 is a side view of a distal end portion of the delivery assembly of FIG. 25, depicting the prosthetic heart valve fully disposed within the delivery capsule.

FIGS. 27-32 depict various portions of an exemplary implantation procedure in which the delivery assembly of FIG. 25 is used.

FIG. 33 is a perspective view of another exemplary prosthetic heart valve, depicting a frame of the prosthetic heart valve in a radially-expanded configuration and without a valve structure thereto.

FIG. 34 is a perspective view of the prosthetic heart valve of FIG. 33, depicting the frame of the prosthetic heart valve in a radially-compressed configuration and without the valve structure coupled thereto.

FIG. 35 is a perspective view of another exemplary prosthetic heart valve, depicting a frame of the prosthetic heart valve in a radially-expanded configuration.

### DETAILED DESCRIPTION

### General Considerations

It should be understood that the disclosed examples can be adapted for delivering and implanting prosthetic heart valves in any of the native annuluses of the heart (e.g., the aortic, pulmonary, mitral, and tricuspid annuluses), and can be used with any of the various delivery devices for delivering the prosthetic heart valve using any of a number of delivery approaches (e.g., retrograde, antegrade, transseptal, transseptal, transventricular, transatrial, etc.). Although the examples of delivery apparatuses disclosed herein are described in the context of being to implant a prosthetic heart valve, the delivery apparatuses can be used to deliver and implant any of various medical implants within the body, including, but not limited to, venous valves, stents, grafts, heart valve repair devices, etc.

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not excluded the presence of intermediate elements between the coupled or associated items absent specific contrary language.

As used in this application, the term “and/or” used between the last two of a list of elements any one or more of the listed elements. For example, the phrase “A, B, and/or C” means “A,” “B,” “C,” “A and B,” “A and C,” “B and C,” or “A, B, and C.”

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while the distal motion of the device is motion of the device away from the user and toward the implantation site (e.g., into the patient's body). The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such has the longitudinal axis of the prosthetic heart valve).

### Introduction to the Disclosed Technology

Prosthetic devices (e.g., stents and prosthetic valves) may include a non-smooth outer surface. For example, stent or a frame of a prosthetic valve can include a lattice type structure with a plurality of struts which form cells. Additionally or alternatively, some prosthetic valves comprise projections extending outwardly from the valve. These projects can, for example, include portions of a valvular structure (e.g., leaflet commissures), portions of an expansion mechanism, valve anchoring members, and/or paravalvular leakage (PVL) reduction elements (e.g., a skirt). When radially compressed and loaded into a delivery capsule (e.g., a sheath) of a delivery apparatus, one or more components forming the non-smooth outer surface the prosthetic valve can contact the inner surface of the delivery capsule. To deploy the prosthetic valve, the delivery capsule is retracted from the prosthetic valve and/or the prosthetic valve is advanced from delivery capsule. The frictional forces caused by the contact and the relative movement between the prosthetic valve and the delivery capsule can, in some instances, result in the prosthetic valve sticking and/or jumping as the prosthetic valve is deployed from the delivery capsule. This can, for example, result in relatively high forces to be used to deploy the prosthetic valve and/or result in the prosthetic valve being misaligned and undesirably positioned relative to an implantation location. The erratic nature can also reduce predictability and repeatability of a delivery procedure. In some instances, an errantly positioned valve can interfere with native anatomy (e.g., coronary ostia), which can result in undesirable patient outcomes.

When positioning the prosthetic valve relative to the native anatomy, the prosthetic valve may need to be rotated relative to the native tissue (e.g., to avoid blocking the coronary ostia). This can be accomplished, for example, by rotating the shaft of the delivery apparatus to which the prosthetic valve is attached and/or by rotating the delivery capsule in which the prosthetic valve is disposed. In some instances, however, the prosthetic valve and the delivery capsule do not rotate together. This can be caused by slippage between the prosthetic valve and the delivery capsule and result in relative rotation therebetween and ultimately undesirable prosthetic valve orientation.

Accordingly, there is a need for improved delivery capsules that can reduce the frictional forces acting on the prosthetic valve during delivery, as well as provide a mechanism by which the prosthetic valve can be rotationally oriented prior to capsule retraction and valve expansion.

Described herein are delivery apparatus and methods for implanting prosthetic heart valves, and/or other expandable medical devices. More specifically, the disclosed delivery apparatus can comprise one or more inner guide rails extending radially inwardly from and axially along the inner surface of the delivery capsule. The inner guide rails can be configured to contact less than the entire outer surface of a prosthetic valve. Since the disclosed inner guide rails engage a relatively small portion of the outer surface of the prosthetic valve compared to a typical delivery capsule, the disclosed delivery capsules reduce axial friction between the prosthetic valve and the delivery capsule during valve deployment. This can, among other things, reduce the forces required to deploy the prosthetic valve from the delivery capsule and/or can help to promote smooth, controlled, and/or less erratic valve deployment.

In some examples, the inner guide rails can comprise axially-extending recesses formed therein. The recesses can be configured to receive a portion of the prosthetic valve (e.g., a portion of a valve frame, an actuation mechanism, and/or a portion of the valvular structure (e.g., leaflet commissure), etc.). In this manner, the recesses in the delivery capsule allow relative axial movement between the prosthetic valve and the delivery capsule (e.g., during deployment and/or retrieval of the prosthetic valve) and also restrict relative rotational movement between the prosthetic valve and the delivery capsule (e.g., during positioning the prosthetic valve relative to the native anatomy). This can, for example, allow the delivery capsule to be used to orient the prosthetic valve relative to the native anatomy during an implantation procedure.

In lieu of or in addition to the inner guide rails and/or recesses, the disclosed delivery capsules can, in some examples, comprise one or more outer guide rails extending radially outwardly from and axially along the outer surface of the delivery capsule. The outer guide rails can be configured to contact less than the entire inner surface of an introducer (e.g., which is inserted into the patient's vasculature to provide an access point for the delivery apparatus and prosthetic valve). Since the disclosed outer guide rails engage a relatively small portion of the inner surface of the introducer compared to a typical delivery capsule, the disclosed delivery capsules reduce axial friction between the prosthetic valve and the introducer when the delivery capsules are passing through the introducer. This can, among other things, reduce the forces required to deploy the prosthetic valve from the delivery capsule and/or can help to promote smooth, controlled, and/or less erratic valve deployment.

Additional information about the disclosed delivery capsules, as well as exemplary delivery apparatus and prosthetic valves, is provided below.

### Examples of the Disclosed Technology

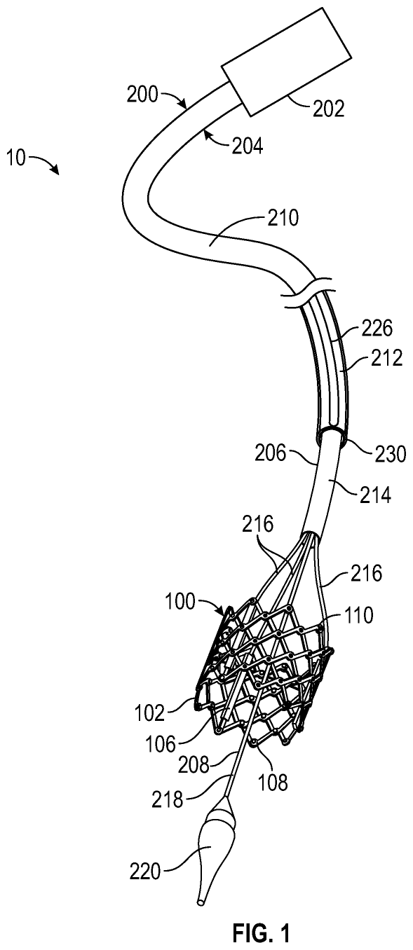

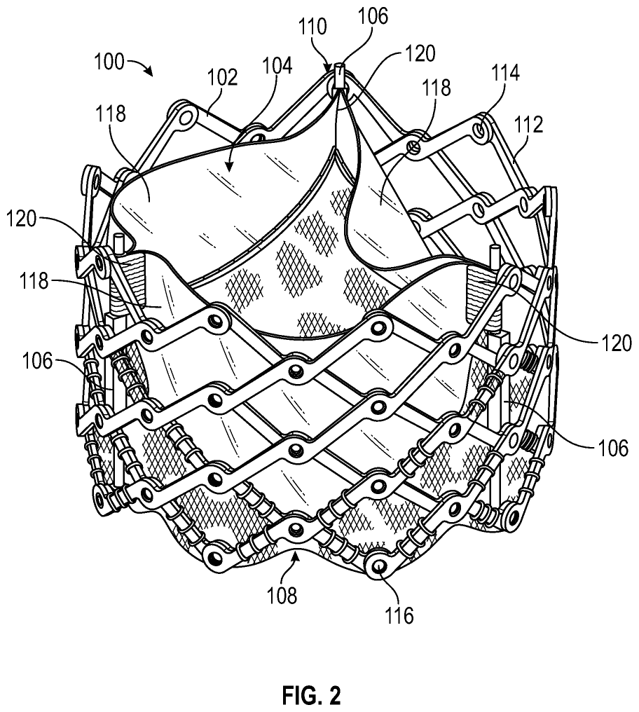

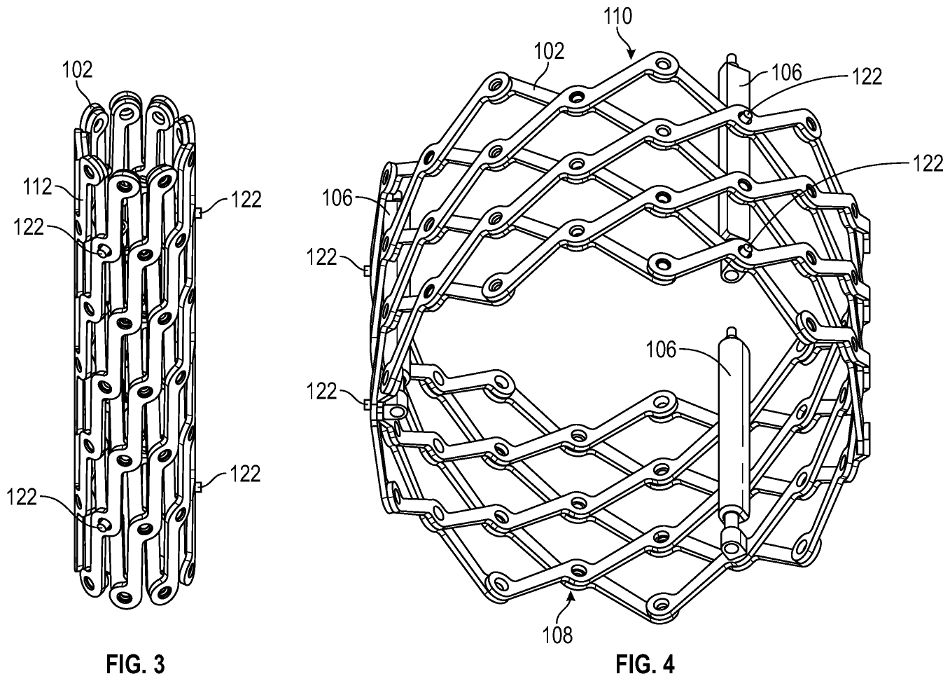

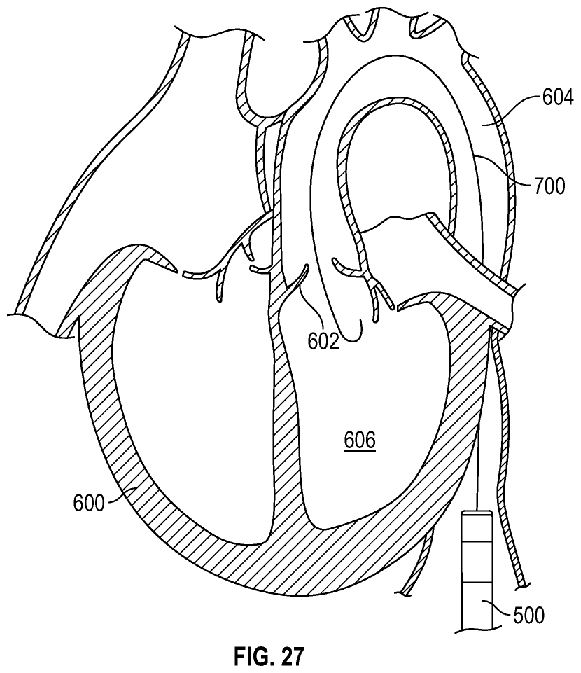

FIG. 1 depicts a delivery assembly 10, according to one example. In the illustrated example, the delivery assembly 10 comprises a prosthetic heart valve 100 and a delivery apparatus 200. The prosthetic heart valve 100 can be releasably coupled to the distal end portion of the delivery apparatus. The prosthetic heart valve 100 can be radially compressed to a delivery configuration (e.g., FIG. 3) and positioned within a delivery capsule of the delivery apparatus 200. The delivery apparatus 200 can be used to insert the prosthetic heart valve 100 into a patient's vasculature and to position the prosthetic heart valve 100 relative to the patient's native anatomy. The delivery apparatus 200 can also be used to deploy the prosthetic heart valve 100 from the delivery capsule and (in some instances) to radially expand the prosthetic heart valve from the delivery configuration to a deployed, functional configuration (e.g., FIGS. 2 and 4). An exemplary delivery procedure is described further below with reference to FIGS. 27-32. Additional details of the prosthetic heart valve 100 and the delivery apparatus 200 are also provided immediately below.

It should be noted at the outset that, although the exemplary prosthetic heart valves and delivery apparatus disclosed herein are primarily directed to transcatheter aortic valve implantation (TAVI), the technology and methods disclosed herein can be used and/or readily adapted for use in various other implantation locations and/or with various types of prosthetic devices. For example, the delivery apparatus disclosed herein can be configured for implanting a prosthetic valve at the native mitral, pulmonary, and/or tricuspid valve regions. Additionally, the delivery apparatus disclosed herein can be used with stents or other types of prosthetic devices that are disposed in a delivery capsule during a portion of an implantation procedure.

FIG. 2 depicts the prosthetic heart valve 100, which is an exemplary mechanically-expandable prosthetic heart valve. The prosthetic heart valve 100 comprises three main components: a frame 102, a valve structure 104, and a plurality of actuation members 106. In FIGS. 1 and 3-4, the valve structure 104 is omitted to better illustrate the frame 102 and the actuation members 106. The frame 102, which can also be referred to as “a stent” or “a support structure,” is configured for supporting the valve structure 104 and for securing the prosthetic heart valve 100 to native tissue (e.g., a native heart valve annulus). Referring again to FIG. 2, the valve structure 104 can be coupled to the frame 102 and/or to the actuation members 106. The valve structure 104 is configured to allow blood flow through the prosthetic heart valve 100 in one direction (i.e., antegrade) and to restrict blood flow through the prosthetic heart valve 100 in the opposite direction (i.e., retrograde). In this manner, the prosthetic heart valve 100 comprise an inflow end 108 and an outflow end 110. The actuation members 106 are coupled to the frame 102 are configured to adjust expansion of the frame 102 to a plurality of configurations including one or more functional or expanded configurations (e.g., FIGS. 1-2 and 4), one or more delivery or compressed configurations (e.g., FIG. 3), and/or one or more intermediate configurations between the functional and delivery configurations.

Referring to FIGS. 3-4, the frame 102 of the prosthetic heart valve 100 includes a plurality of interconnected struts 112 arranged in a lattice-type pattern. As depicted in FIG. 4, when the frame 102 is in a radially-expanded configuration, the struts 112 of the frame 102 extend diagonally relative to a longitudinal axis of the prosthetic heart valve 100. In other configurations, the struts 112 of the frame 102 can be offset by a different amount than the amount depicted in FIG. 4. For example, FIG. 3 depicts the frame 102 in a radially-compressed configuration (which is also referred to herein as “a delivery configuration”). In the delivery configuration, the struts 112 of the frame 102 extend parallel (or at least substantially parallel) to the longitudinal axis of the prosthetic heart valve 100.

To facilitate movement between the expanded and compressed configurations, the struts 112 of the frame 102 are pivotably coupled to one another at one or more pivot joints 114. For example, the struts can comprise openings that are configured to receive pivot elements 116 (e.g., rivets, pins, tabs, etc.). In some examples, each of the two pivotably-connected struts can comprise an opening, and the pivot element can extend through the opening of both struts. In other examples, a first strut of two pivotably-connected struts can comprise the pivot element (e.g., fixedly attached thereto or integrally formed thereon), and a second strut of the two pivotably-connected struts strut can comprise an opening configured to receive the pivot element of the first strut. In any event, the pivot joints 114 allow the struts 112 to pivot relative to one another as the frame 102 moves between the radially-expanded configuration and the radially-compressed configuration.

The frame 102 of the prosthetic heart valve 100 can be made of any suitable materials, including biocompatible metals and/or biocompatible polymers. Exemplary biocompatible metals from which the frame can be formed include stainless steel, cobalt chromium alloy, and/or nickel titanium alloy (which can also be referred to as “NiTi” or “nitinol”).

With reference to FIG. 2, the valve structure 104 of the prosthetic heart valve 100 can comprise a plurality of leaflets 118 that collectively form a leaflet assembly. The leaflets 118 can be arranged to form commissures 120 (e.g., pairs of adjacent leaflets), which can, for example, be mounted to respective actuation members 106 and/or to the frame 102.

The leaflets 118 of the prosthetic heart valve 100 can be made of a flexible material such that the leaflets 118 can open and close to regulate the one-way flow of blood through the valve structure 104. For example, the leaflets 118 can be made from in whole or in part, biological material, bio-compatible synthetic materials, and/or other such materials. Suitable biological material can include, for example, bovine pericardium, porcine pericardium, equine pericardium, ovine pericardium, etc.

Further details regarding prosthetic heart valves, including the manner in which the valve structure 104 can be coupled to the frame 102 of the prosthetic heart valve 100, can be found in U.S. Pat. Nos. 6,730,118, 7,393,360, 7,510,575, 7,993,394, and 8,652,202, and U.S. Publication Nos. 2018/0153689 and 2018/0325665, which are incorporated by reference herein.

As depicted in FIG. 4, the actuation members 106 of the prosthetic heart valve 100 are mounted to and spaced circumferentially around the interior of the frame 102. In the illustrated example, the prosthetic heart valve 100 comprises three actuation members 106. It should be noted that in other examples the prosthetic heart valve 100 can comprise fewer (e.g., 1-2) or more (e.g., 4-15) than three actuation members.

The actuation members 106 are configured to, among other things, radially expand and/or radially compress the frame 102. For this reason, the actuation members 106 can be referred to as “expansion mechanisms.” In some examples, the actuation members 106 can also be configured to lock the frame 102 at a desired expanded configuration. Accordingly, the actuation members 106 can also be referred to as “lockers” or “locking mechanisms.”

The actuation members 106 can be configured to form a releasable connection with one or more respective actuation shafts of a delivery apparatus (e.g., FIG. 1). This can be accomplished in various ways, such as a threaded connection, a male/female mating connection, and/or various other means for releasably connecting.

As depicted in FIGS. 3-4, each of the actuation members 106 of the prosthetic heart valve 100 can be coupled to the frame 102 via one or more fasteners 122 (e.g., rivets or pins). In some examples, each of the fasteners 122 can extend from the actuation members 106, through a respective aperture of the struts 112, and radially outwardly away from an outer surface of the frame 102. As such, the fasteners 122 project or protrude beyond the outer surface of the frame 102 in a manner that can cause unwanted friction between the prosthetic heart valve 100 and components of the delivery apparatus 200, thereby impeding the implantation of the prosthetic heart valve.

For example, during implantation, a delivery capsule of a conventional delivery apparatus extends over and contacts the fasteners 122 and outer surface of the prosthetic heart valve when it is in the delivery configuration. Once positioned at or adjacent an implantation location, the delivery capsule is retracted from the prosthetic heart valve 100 and/or the prosthetic heart valve 100 is advance out of the delivery capsule. The relative movement between the prosthetic heart valve 100 and the delivery capsule generates friction between the prosthetic heart valve 100 and the inner surface of the delivery capsule with which the valve is in contact. In particular, the fasteners 122 of prosthetic heart valve dragging along the delivery capsule can generate friction. This friction can, for example, result in the need to use relatively high forces to deploy the prosthetic heart valve. It can also result in unwanted movement (e.g., axial and/or rotational) during deployment. The erratic movement of the prosthetic heart valve may be referred to as “jumping” or “shifting.” Unwanted and/or erratic movement can, for example, result in an undesirably placed prosthetic heart valve. For example, the prosthetic heart valve may obstruct or interfere with native anatomy (e.g., the coronary ostia).

Further, conventional delivery capsules are not configured to realign (e.g., axially or rotationally) the prosthetic heart valve once misalignment occurs.

As another issue, conventional delivery capsules may encounter relatively high frictional forces when passing through an outer shaft (e.g., an introducer) of the delivery assembly. As a result, the friction between the delivery capsule and the outer shaft can often require the medical practitioner and/or the delivery apparatus to apply a relatively high force to advance the delivery capsule through the generally narrower lumen of the outer shaft.

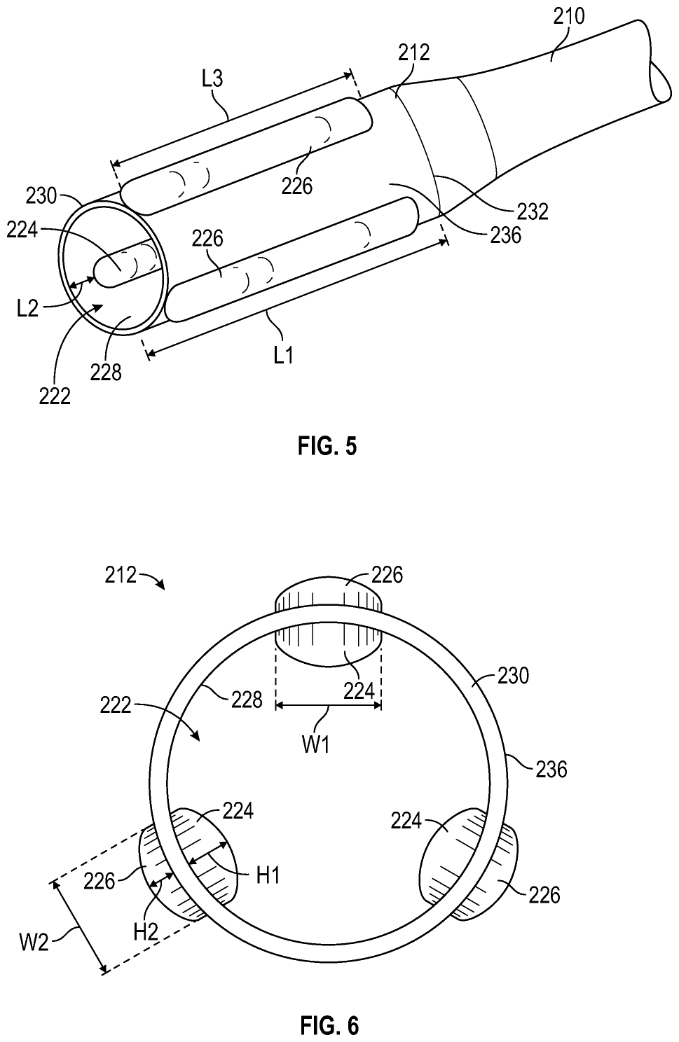

Described herein are delivery capsules (see, e.g., FIG. 5) configured to reduce the friction created between the delivery capsule and the prosthetic heart valve and/or an outer shaft (e.g., an introducer of the delivery assembly. The disclosed delivery capsules can, for example, include inner guide rails configured to reduce the contact area (and thus the friction) between the prosthetic heart valve and the delivery capsule. The inner guide rails can additionally or alternatively be used to rotate the prosthetic heart valve relative to the native anatomy and/or other components of the delivery apparatus (e.g., during an implantation procedure). In lieu of or in addition to the inner guide rails, the delivery capsules disclosed herein can include outer guide rails configured to reduce the contact area (and thus the friction) between the delivery capsule and a shaft through which the delivery capsule passes (e.g., an introducer).

FIG. 1 schematically depicts the delivery apparatus 200, as one example. The delivery apparatus 200 comprises a handle 202, a delivery catheter 204, an implant catheter 206, and a guide wire catheter 208. The implant catheter 206 extends axially through the delivery catheter 204, and the guide wire catheter 208 extends axially through the implant catheter 206 (and the delivery catheter 204). Each of the catheters 204, 206, 208 is movable relative to each other (e.g., axially and/or rotationally). The proximal end portions of the catheters 204, 206, 208 are coupled to the handle 202. As schematically depicted, each catheter is coupled to the handle 202. In other examples, the delivery apparatus can comprise a plurality of handles, and the proximal end portion of each catheter can be coupled to a respective handle.

Generally speaking, the delivery catheter 204 is configured to cover the prosthetic heart valve as the delivery assembly (i.e., the delivery apparatus and the prosthetic heart valve) is inserted into a patient's vasculature and advanced to an implantation location. The implant catheter 206 is configured to be releasably coupled to the prosthetic heart valve and to manipulate the expansion and/or contraction of the prosthetic heart valve at the implantation location. The guide wire catheter 208 is configured to track over a guide wire (which is inserted prior to insertion of the delivery apparatus 200) and route the delivery apparatus 200 to the implantation location.

Referring still to FIG. 1, the delivery catheter 204 comprises an outer shaft 210 and a delivery capsule 212 coupled to the distal end portion of the outer shaft 210. In some examples, the outer shaft 210 and the delivery capsule 212 can be integrally formed (e.g., co-molded) as a single, unitary component. In other examples, the outer shaft 210 and the delivery capsule 212 can be formed as separate components that are coupled together (e.g., over-molding, bonding, adhesive, fasteners, and/or other means for coupling). Additional details about the delivery capsule 212 are provided below.

The implant catheter 206 comprises a main shaft 214 and one or more actuation shafts 216 extending through the main shaft 214. The actuation shafts 216 can be releasably coupled to the actuation members 106 of the prosthetic heart valve 100 and can be used to manipulate the prosthetic heart valve 100. The guide wire catheter 208 comprises a guide wire shaft 218 and a nosecone 220 coupled to the distal end portion of the guide wire shaft 218.

Additional details about handles, delivery catheters, implant catheters, the guide wire catheters, releasably coupling the prosthetic heart valve to the delivery apparatus, and/or using the delivery apparatus to manipulate the prosthetic heart valve can be found, for example, in U.S. Pat. No. 10,973,634, U.S. Publication No. 2018/0153689, and International Publication No. WO 2021/188476, which are incorporated by reference herein.

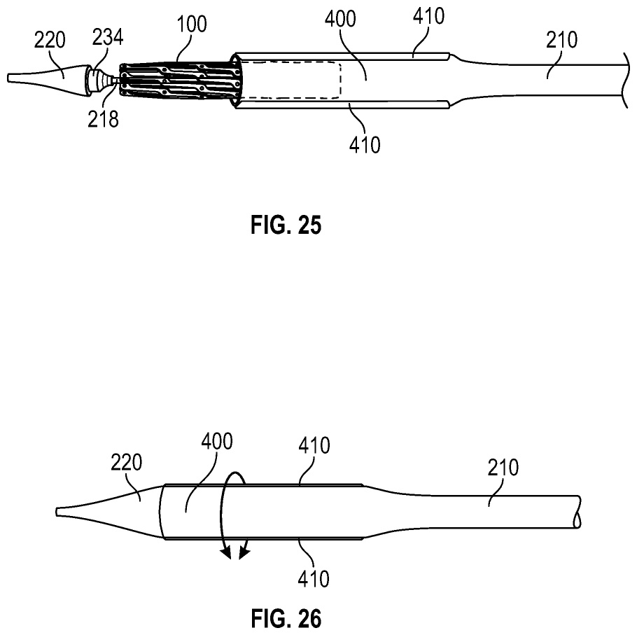

Turning now to FIG. 5, the delivery capsule 212 of the delivery apparatus 200 comprises a lumen 222, one or more inner guide rails 224 (which also can be referred to as “inner positioning ribs”), and one or more outer guide rails 226 (which also can be referred to as “outer positioning ribs”). The lumen 222 is configured to receive and/or retain the prosthetic heart valve 100 in the radially-compressed configuration (see, e.g., FIGS. 25-26). The inner guide rails 224 are configured to contact the outer surface of the prosthetic heart valve. The outer guide rails 226 are configured to contact the inner surface of lumen disposed radially outwardly from the delivery capsule (e.g., the inner surface of an introducer).

The lumen 222 of the delivery capsule 212 is defined primarily by an inner surface 228 of the delivery capsule 212. The lumen 222 comprises an axial length L1, which is similar to the axial length of the prosthetic heart valve 100 in the radially-compressed configuration. The lumen 222 can also receive the proximal end portion of the nosecone 220 (see FIGS. 25-26).

As depicted in FIG. 6, the inner guide rails 224 of the delivery capsule 212 extend radially inwardly from the inner surface 228 of the delivery capsule 212 and are circumferentially spaced apart relative to each other. In the illustrated example, the delivery capsule 212 comprises three inner guide rails 224 circumferentially spaced equidistant from one another. In other examples, a delivery capsule can comprise less or more than three (e.g., 1-2 or 4-15) inner guide rails and/or be circumferentially spaced at equal or unequal intervals.

Referring again to FIG. 5, the inner guide rails 224 can extend axially from at or adjacent the distal end 230 of the delivery capsule 212 toward or to the proximal end 232 of the delivery capsule 212. In some instances, the inner guide rails can extend along the entire length of the lumen (i.e., from the distal end 230 to the proximal end 232 such that the inner guide rails have an axial length equal to L1). In other instances, the inner guide rails can extend along less than the entire length of the lumen. For example, in the illustrated example, the inner guide rails 224 extend less than the entire length of the lumen 222 and are axially spaced from the distal end 230 of the delivery capsule 212 by a distance L2. The distance L2 can (in some instances) correspond to the axial length of a proximal shoulder 234 of the nosecone 220 (see FIG. 25).

As mentioned above, the inner guide rails 224 are configured to contact the outer surface of the prosthetic heart valve and can space the prosthetic heart valve from the inner surface 228 of the delivery capsule 212 (or at least reduce the extent in which the prosthetic heart valve contacts the inner surface 228). Since the inner guide rails 224 contact only a relatively small portion of the total circumferential area of the prosthetic heart valve, the inner guide rails 224 can, thereby reduce the friction between the prosthetic heart valve and the delivery capsule. As such, less force is needed to deploy the prosthetic heart valve and deployment can be more consistent and/or predictable (e.g., it reduces valve “jumping”).

As depicted in FIGS. 5-6, the outer guide rails 226 of the delivery capsule 212 extend radially outwardly from an outer surface 236 of the delivery capsule 212 and are circumferentially spaced apart relative to each other. In the illustrated example, the delivery capsule 212 comprises three outer guide rails 226 circumferentially spaced equidistant from one another. In other examples, a delivery capsule can comprise less or more than three (e.g., 1-2 or 4-15) outer guide rails and/or circumferentially spaced at equal or unequal intervals.

Referring to FIG. 5, the outer guide rails 226 can extend axially from at or adjacent the distal end 230 of the delivery capsule 212 toward or to the proximal end 232 of the delivery capsule 212 such that the outer guide rails 226 comprise an axial length L3. In some instances, the outer guide rails can extend along the entire length of the lumen (i.e., L3 is equal to L1). In other instances, the outer guide rails can extend along less than the entire length of the lumen. For example, in the illustrated example, the outer guide rails 226 extend less than the entire length of the lumen 222 and are axially spaced from the proximal end 232 of the delivery capsule 212. Additionally or alternatively, the outer guide rails can be axially spaced from the distal end 230 of the delivery capsule.

The outer guide rails 226 are configured such that when the delivery capsule 212 is inserted through a lumen (e.g., of an introducer) the outer guide rails 226 contact the inner surface of the introducer. Due to the relatively small amount of surface area of the outer guide rails 226, the friction between the delivery capsule and the introducer is reduced compared to typical delivery capsules in which all or substantially all of the outer surface of the delivery capsule engages the inner surface of the introducer.

The inner guides rails and the outer guide rails can comprise various sizes. The inner guide rails 224 comprise a max height H1 and a max width W1. The outer guide rails 226 comprise a max height H2 and a max width W2. It should be noted that the heights H1 and H2 and the widths W1 and W2 of the guide rails depicted in FIG. 6 are merely exemplary. In other examples, the heights H1 and H2 and/or the widths W1 and W2 of the guide rails can be greater or less than those depicted. For example, the height H1 of the inner guide rails 224 can be configured such that the inner guide rails 224 contact the outer surface of a prosthetic heart valve and there is a radially- and/or circumferentially-extending gap between the outer surface of the prosthetic heart valve and the inner surface 228 of the delivery capsule at locations between adjacent pairs of the inner guide rails 224. In other examples, the height H1 of the inner guide rails 224 can be configured such that the inner guide rails 224 contact the outer surface of a prosthetic heart valve and there is not a radially- and/or circumferentially-extending gap between the outer surface of the prosthetic heart valve and the inner surface 228 of the delivery capsule at locations between adjacent pairs of the inner guide rails 224. Despite a lack of a gap, the inner guide rails can still reduce the extent in which the outer surface of the prosthetic heart valve engages the inner surface 228 of the delivery capsule. In either instance, the inner guide rails can, for example, reduce friction between the prosthetic heart valve and the delivery capsule.

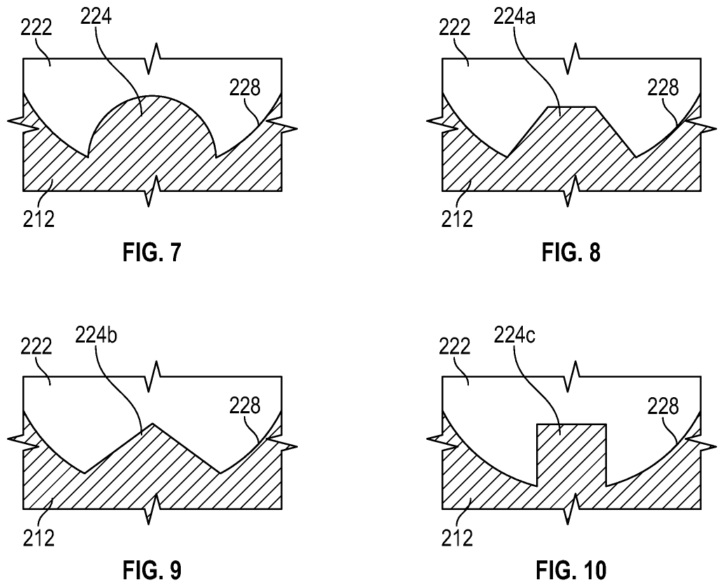

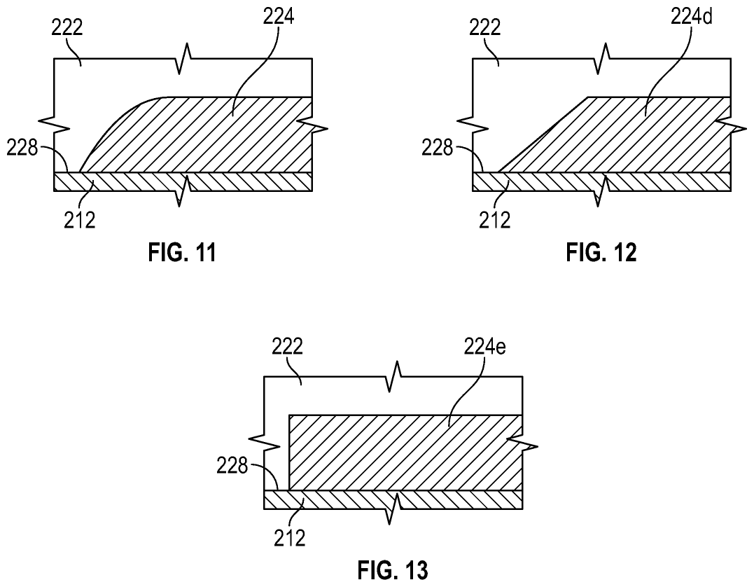

Referring now to FIGS. 7-13, the inner guides rails and the outer guide rails can comprise various shapes. For example, FIGS. 7-10 depict the cross-sectional profile of several exemplary inner guide rails taken in a plane perpendicular to a central longitudinal axis of the delivery capsule, and FIGS. 11-13 depict the partial cross-sectional profile of several exemplary inner guide rails taken in a plane parallel to the central longitudinal axis of the delivery capsule. More specifically, FIG. 7 depicts one of the inner guide rails 224 comprising a semi-circular cross-sectional profile taken in a plane perpendicular to the central longitudinal axis of the delivery capsule 212. FIG. 8 depicts an inner guide rail 224a comprising a trapezoidal cross-sectional profile taken in a plane perpendicular to the central longitudinal axis of the delivery capsule 212. FIG. 9 depicts an inner guide rail 224b comprising a triangular cross-sectional profile taken in a plane perpendicular to the central longitudinal axis of the delivery capsule 212. FIG. 10 depicts an inner guide rail 224c comprising a rectangular cross-sectional profile taken in a plane perpendicular to the central longitudinal axis of the delivery capsule 212. FIG. 11 depicts one of the inner guide rails 224 comprising a curved or rounded cross-sectional profile taken in a plane parallel to the central longitudinal axis of the delivery capsule 212. FIG. 12 depicts an inner guide rail 224d comprising a tapered or angled cross-sectional profile taken in a plane parallel to the central longitudinal axis of the delivery capsule 212. FIG. 13 depicts an inner guide rail 224e comprising a rectangular cross-sectional profile taken in a plane parallel to the central longitudinal axis of the delivery capsule 212. The outer guide rails can comprise shapes similar to those described and/or depicted for the inner guide rails. Various other shapes can be used for the inner guide rails and/or the outer guide rails.

In the illustrated example, all of the inner guide rails 224 comprises a similar size and shape. In other examples, one or more of the inner guide rails can comprise a different size and/or shape than one or more other inner guide rails. Similarly, in the illustrated example, all of the outer guide rails comprises a similar size and shape. In other examples, one or more of the outer guide rails can comprise a different size and/or shape than one or more other outer guide rails. Also, in the illustrated example, the inner guide rails 224 comprise a similar size, shape, and/or quantity as the outer guide rails 226. In other examples, one or more of the inner guide rails can comprise a different size, shape, and/or quantity than the outer guide rails.

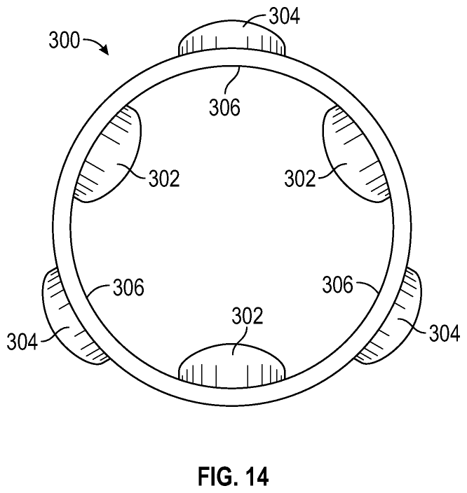

The inner guide rails and the outer guide rails can be configured such that inner guide rails and the outer guide rails are circumferentially aligned and/or offset relative to each other. For example, the inner guide rails 224 and the outer guide rails 226 of the delivery capsule 212 are circumferentially aligned, as depicted in FIG. 6. FIG. 14 depicts another exemplary delivery capsule 300 comprising a plurality of inner guide rails 302 and a plurality of outer guide rails 304, which are circumferentially offset relative to the inner guide rails 302. This configuration can, in some examples, allow the delivery capsule 300 to deflect radially inwardly when a radially compressive force is exerted on the outer guide rails 304 (e.g., when the delivery capsule is passing through and introducer) because of the space or gap between an inner surface 306 of the delivery capsule 300 and the outer surface of a prosthetic heart valve disposed in the delivery capsule 300 at circumferential locations between the inner guide rails 302 of the delivery capsule 300.

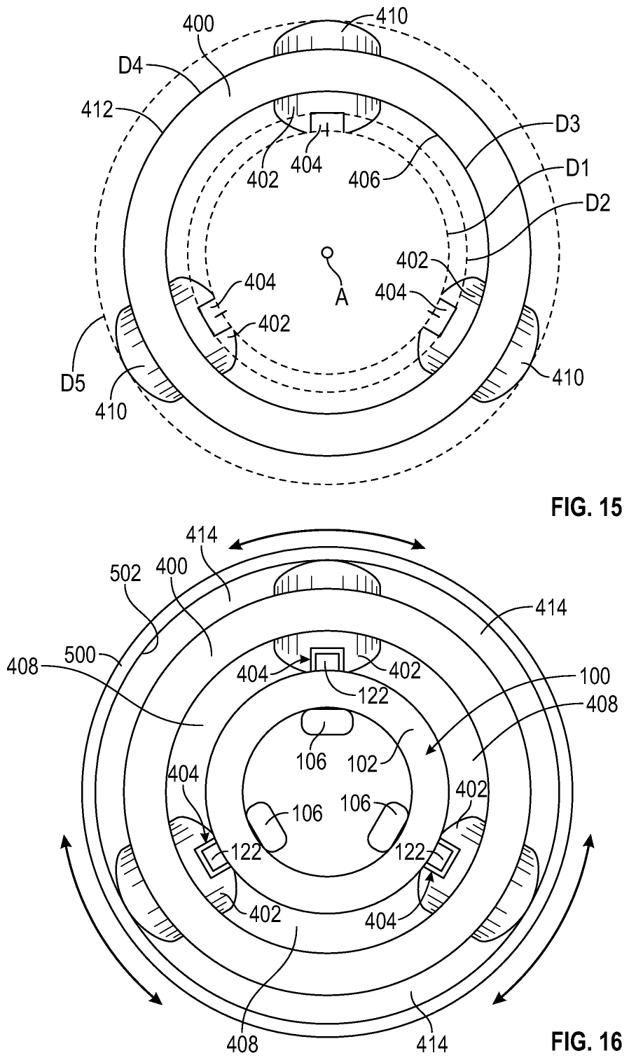

FIGS. 15-16 depict a delivery capsule 400, according to another example. The delivery capsule 400 is generally configured similar to the delivery capsule 212. One difference between the delivery capsule 400 and the delivery capsule 212, however, is that inner guide rails 402 of the delivery capsule 400 comprise recesses, or grooves 404. The recesses 404 can be configured to receive a portion or a member of a prosthetic implant. For example, as depicted in FIG. 16, the recesses 404 can receive the fasteners 122 of the prosthetic heart valve 100 (which is shown schematically in FIG. 16). As such, the delivery capsule 400 and the prosthetic heart valve 100 mate such that the prosthetic heart valve can move axially (e.g., proximal and/or distal) relative to the delivery capsule and such that the prosthetic heart valve and the delivery capsule move rotationally (e.g., clockwise and/or counterclockwise) together. In some instances, the mating between the prosthetic heart valve and delivery capsule may be referred to as “a keyed connection.” Configuring the delivery capsule 400 in this manner can, for example, allow the delivery capsule to be used and/or aid in positioning the prosthetic heart valve rotationally relative to the native anatomy at or adjacent an implantation location.

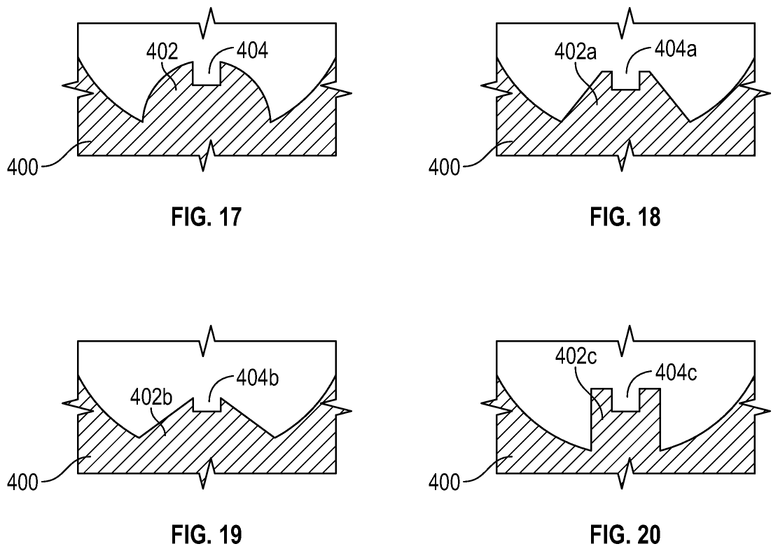

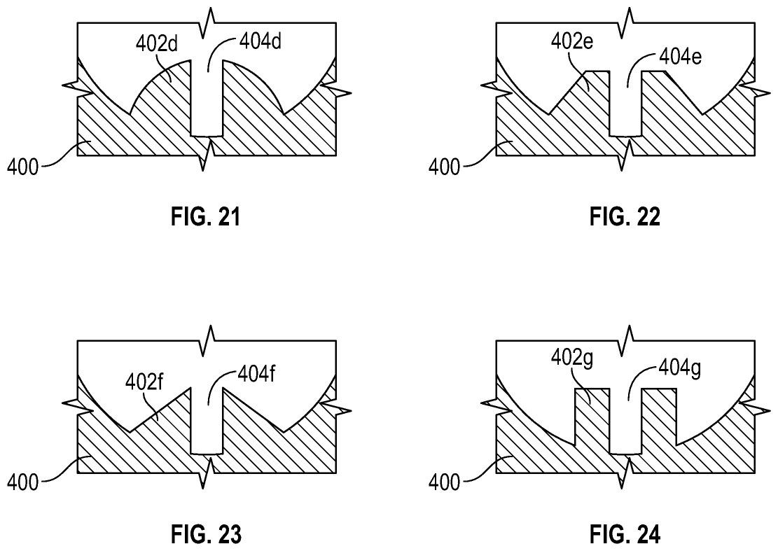

The inner guide rails 402 and/or the recesses 404 can comprise various shapes and sizes. For example, FIGS. 17-24 depict various cross-sectional profiles of inner guide rails taken in a plane perpendicular to the longitudinal axis A (FIG. 15) of the delivery capsule 400. In particular, FIG. 17 depicts one of the inner guide rails 402 comprising a generally semi-circular cross-sectional profile taken in a plane perpendicular to the central longitudinal axis A of the delivery capsule 400. FIG. 18 depicts an inner guide rail 402a comprising a generally trapezoidal cross-sectional profile taken in a plane perpendicular to the central longitudinal axis A of the delivery capsule 400. FIG. 19 depicts an inner guide rail 402b comprising a generally triangular cross-sectional profile taken in a plane perpendicular to the central longitudinal axis A of the delivery capsule 400. FIG. 20 depicts an inner guide rail 402c comprising a generally rectangular cross-sectional profile taken in a plane perpendicular to the central longitudinal axis A of the delivery capsule 400. FIGS. 21-24 are similar to FIGS. 17-20, respectively, except that the depth of the recesses depicted in FIGS. 21-24 is greater than the depth of the recesses depicted in FIGS. 17-20. Due to the depth of the recesses, the configurations depicted in FIGS. 21-24 may, in some instances, be referred to as a pair of inner guide members that are spaced apart from each other by a gap (as opposed to a single inner guide member with a recess formed therein), wherein the gap is the recess.

Referring again to FIG. 15-16, the inner guide rails 402 of the delivery capsule can be configured to accommodate a device therein (e.g., the prosthetic heart valve) comprising a main or primary outer surface with a diameter D1 (which also can be referred to as an “inner rail diameter”). The recesses 404 of the delivery capsule 400 can be configured to accommodate radial protrusions or projections extending from the main outer surface (e.g., fasteners, actuation members, leaflet commissures, PVL skirt, etc.) and having a diameter D2 (which also can be referred to as an “recess diameter”). An inner surface 406 of the delivery capsule 400 can comprise a diameter D3, which is less than the diameter D1. In this manner, the inner guide rails 402 and the recesses 404 of the delivery capsule 400 can space a device (e.g., the prosthetic heart valve 100) from the inner surface 406 of the delivery capsule 400. This creates radially- and circumferentially-extending gaps 408 between the inner surface 406 of the delivery capsule 400 and an outer surface of the prosthetic heart valve 100 at locations between the inner guide rails 402.

In lieu of or in addition to the inner guide rails 402, the delivery capsule 400 can comprise outer guide rails 410. The outer guide rails 410 can extend radially outwardly from an outer surface 412 of the delivery capsule 400. As depicted in FIG. 15, the outer surface 412 of the delivery capsule 400 can comprise a diameter D4, and the outer guide rails 410 can define a diameter D5 (which also can be referred to as an “outer rail diameter”), which is greater than the diameter D4. In this manner, the outer guide rails 410 can create radially- and circumferentially-extending gaps 414 between the outer guide rails 410 and an inner surface 502 of an introducer 500 at locations between the inner guide rails 402, as depicted in FIG. 16.

FIGS. 25-26 depict the prosthetic heart valve 100 being loaded into the delivery capsule 400. More specifically, FIG. 25 depicts the prosthetic heart valve 100 partially loaded into the delivery capsule 400, and FIG. 26 depicts the prosthetic heart valve 100 fully loaded into the delivery capsule 400. Protrusions of the prosthetic heart valve 100 such as the fasteners 122 can be circumferentially aligned with and disposed within the recesses 404 of the inner guide rails 402. As such, rotation of the delivery capsule results in rotation of the prosthetic heart valve, and vice versa.

FIGS. 27-32 schematically depict an exemplary implantation procedure in which a delivery assembly comprising the prosthetic heart valve 100 and the delivery apparatus 200 (with the delivery capsule 400 in lieu of the delivery capsule 212) is used to implant the prosthetic heart valve 100 in a native aortic valve 602 of a heart 600 using a transfemoral delivery procedure.

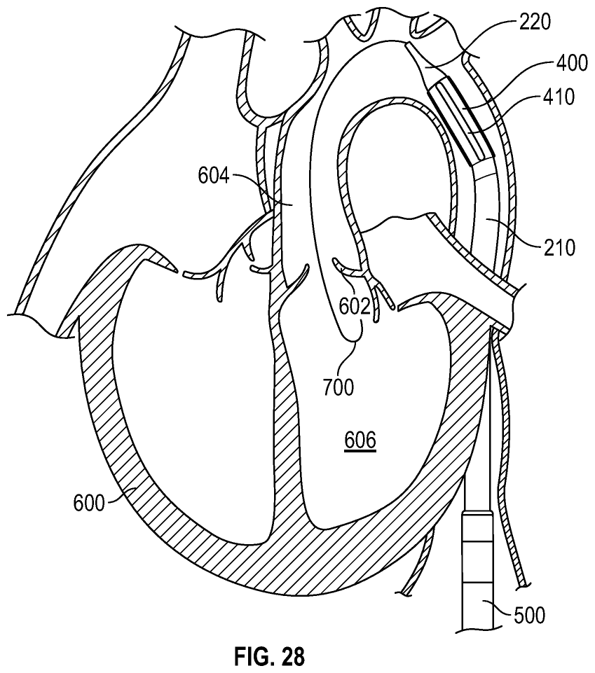

As depicted in FIG. 27, a guide wire 700 is inserted into the patient's vasculature via the introducer 500, and the guide wire 700 extends through the patient's aorta 604 and into the patient's left ventricle 606 using a retrograde approach. As depicted in FIG. 28, the distal end portion of the delivery assembly is advanced over the guide wire 700 and inserted into the patient's vasculature via the introducer 500. The outer guide rails 410 of the delivery capsule 400 can, for example, allow the delivery capsule 400 to pass through the introducer 500 with relatively low forces compared to the forces required for typical delivery capsules.

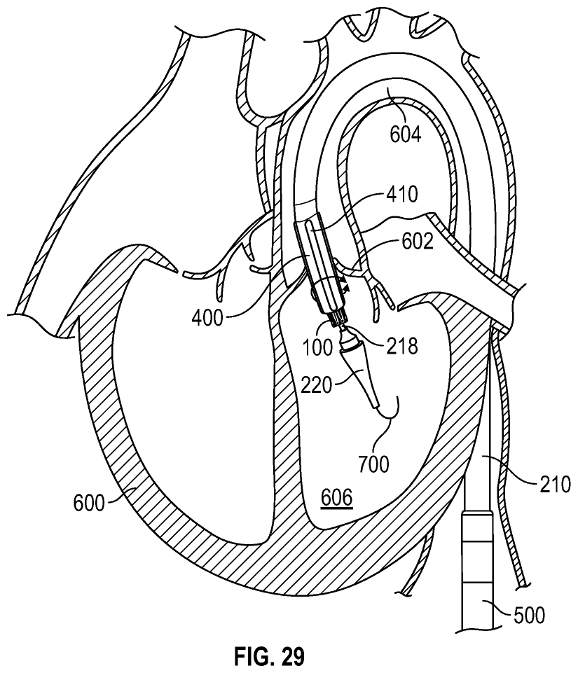

Referring now to FIG. 29, the distal end portion of the delivery assembly is positioned such that the delivery capsule is disposed within the native aortic valve 602. The prosthetic heart valve 100 can be rotationally positioned relative to the native anatomy. For example, the prosthetic heart valve can be positioned such that the coronary ostia are unobstructed (or less obstructed). This can be accomplished by rotating the delivery capsule 400 relative to the native anatomy. Due to a portion of the prosthetic heart valve 100 being disposed in the recesses 404 of the delivery capsule (see, e.g., FIG. 16), the prosthetic heart valve 100 rotates together with the delivery capsule 400. During rotation of the delivery capsule 400 and the prosthetic heart valve 100, the prosthetic heart valve can be fully disposed within the delivery capsule (see, e.g., FIG. 26) or partially disposed within the delivery capsule and partially exposed from the delivery capsule (see, e.g., FIGS. 25 and 29).

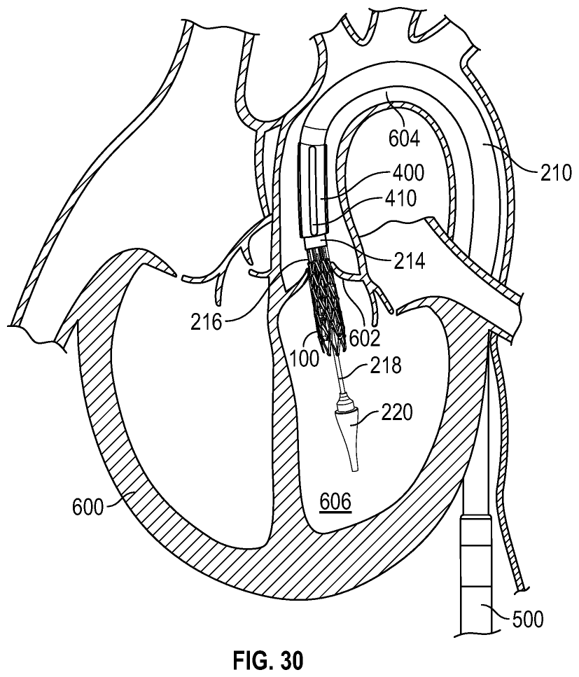

Although FIGS. 29-30 depict the prosthetic heart valve 100 being deployed from the delivery capsule while the delivery capsule and prosthetic heart valve are disposed within the native aortic valve annulus, in other implementations, the delivery capsule can be disposed more superior (e.g., toward the ascending aorta) or more inferior (e.g., toward the left ventricle) during valve deployment.

With the prosthetic heart valve 100 rotationally positioned as desired, the prosthetic heart valve 100 can be fully deployed from the delivery capsule 400. The inner guide rails 402 can, for example, reduce friction between the prosthetic heart valve 100 and the delivery capsule 400 such that the prosthetic heart valve 100 can be deployed from the delivery capsule 400 with relatively lower forces than typical delivery capsules require.

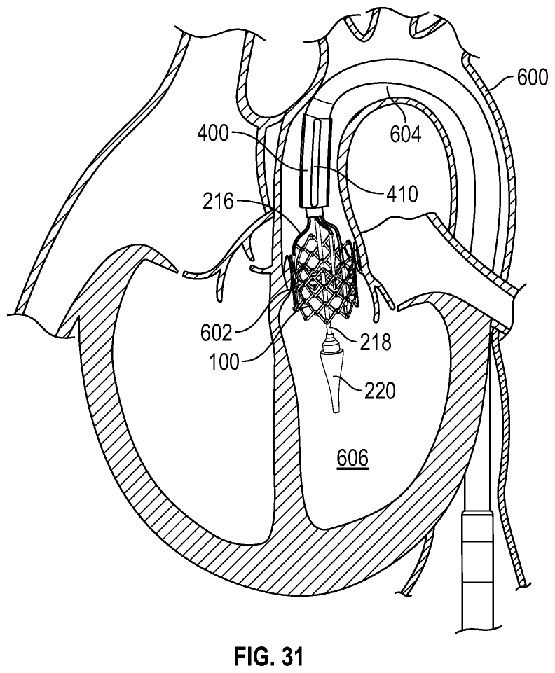

The prosthetic heart valve 100 can be expanded from the radially-compressed configuration to a radially-expanded configuration, as shown for example in FIG. 31. In the illustrated example, the prosthetic heart valve 100 is a mechanically-expandable prosthetic heart valve, which is expanded via the delivery apparatus actuating the actuators of the prosthetic heart valve. In other examples, the prosthetic heart valve can be a self-expandable prosthetic heart valve or a balloon-expandable prosthetic heart valve. In some examples, the prosthetic heart valve can be expanded in a plurality of ways. For example, a prosthetic heart valve may be self-expanding (e.g., due to super-elastic and/or shape-memory properties of the frame) from a delivery configuration to a first expanded configuration and mechanically-expanding (e.g., via actuators) from the first expanded configuration to a second expanded configuration, which is radially larger than the first expanded configuration. As another example, a prosthetic heart valve may be self-expanding (e.g., due to super-elastic and/or shape-memory properties of the frame) from the delivery configuration to the first expanded configuration and balloon-expandable from the first expanded configuration to the second expanded configuration, which is radially larger than the first expanded configuration.

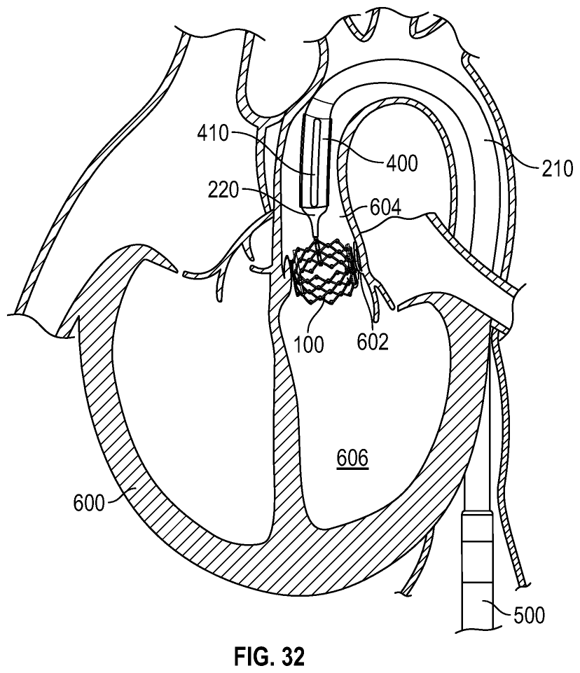

The fully expanded prosthetic heart valve 100 is secure relative to the native anatomy. As such, the prosthetic heart valve 100 can be released from the delivery apparatus 200, and the delivery apparatus 200 can be retracted from the patient's vasculature, as depicted in FIG. 32.

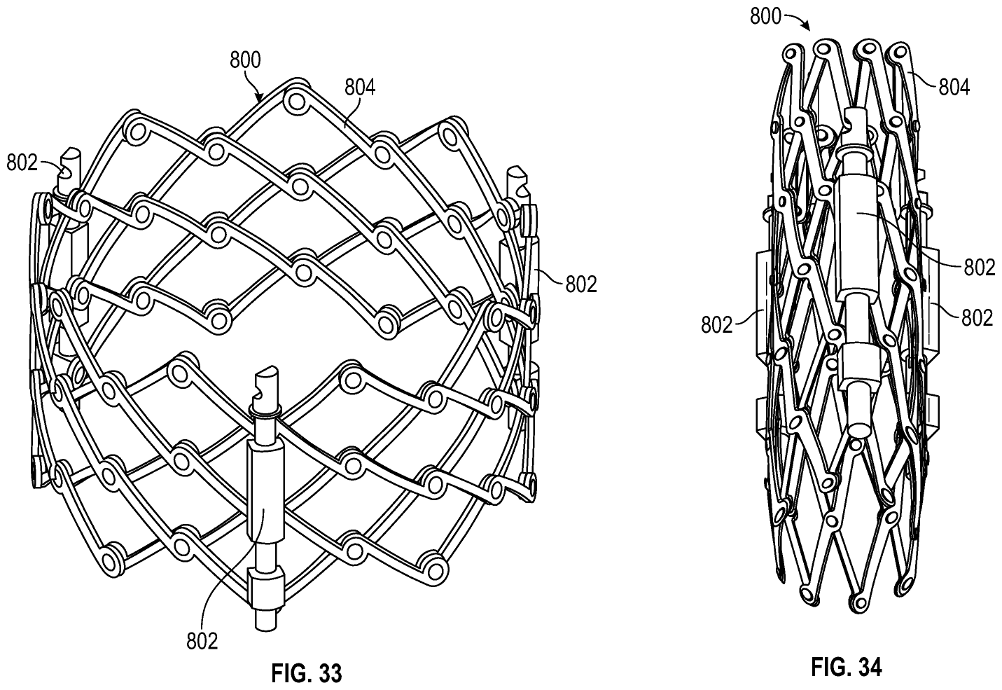

It should be noted that the delivery capsules disclosed herein (e.g., the delivery capsule 212 and/or the delivery capsule 400 can be configured for use with various types of prosthetic heart valve and/or other types of prosthetic implants. FIGS. 33-35 depict several exemplary prosthetic heart valves that can be used with the delivery capsules 212, 400.

For example, FIGS. 33-34 depict an exemplary mechanically-expandable prosthetic heart valve 800. The prosthetic heart valve 800 configured generally similar to the prosthetic heart valve 100, except the prosthetic heart valve 800 comprises actuators 802 disposed on the outside of the frame 804 rather than on the inside of the frame like the actuators 106 of the prosthetic heart valve 100. The prosthetic heart valve 800 can also comprise a valve structure (e.g., including leaflets).

In some examples, the prosthetic heart valve 800 can be radially compressed (e.g., via the actuators 802 and/or a crimping device) and loaded into the delivery capsule 212. In other examples, the prosthetic heart valve 800 can be radially compressed and loaded into delivery capsule 400. In certain examples, the recesses 404 of the delivery capsule 400 can be configured to receive the actuators 802 of the prosthetic heart valve 800 therein.

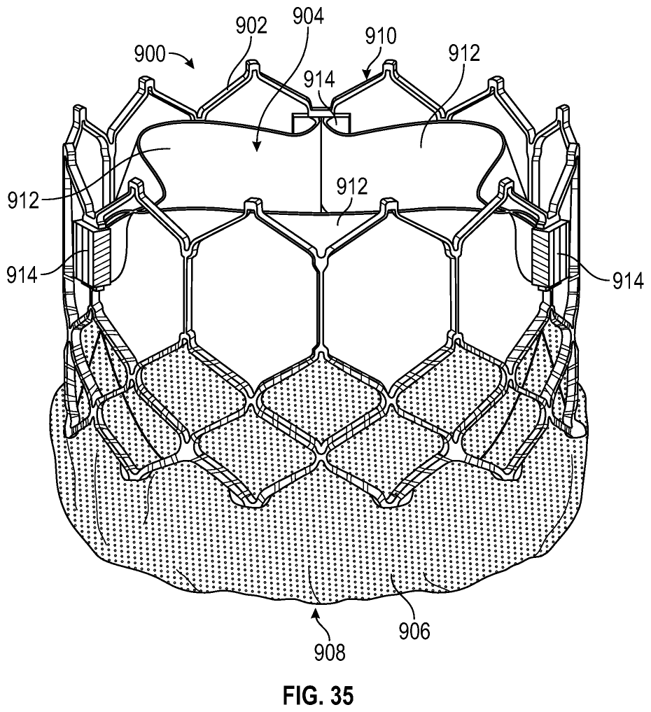

FIG. 35 depicts a prosthetic heart valve 900. The prosthetic heart valve 900 can be self-expanding and/or balloon-expanding. The prosthetic heart valve 900 comprises a frame 902, a valve structure 904, and a sealing structure 906. The frame 902 can be configured for supporting the valve structure 904 and for securing the prosthetic heart valve 900 to the native anatomy. The frame 902 can be formed of a metal comprising stainless steel, cobalt-chromium (e.g., MP35N®), nitinol, and/or other suitable material. The valve structure 904 can be coupled to the frame 902 and is configured to allow blood to flow through the prosthetic heart valve 900 in one direction (i.e., from an inflow end 908 to an outflow end 910). The valve structure 904 can comprise a plurality of leaflets 912, and the leaflets 912 can form commissures 914 where each leaflet pair meets.

In some examples, the prosthetic heart valve 900 can be radially compressed (e.g., via a crimping device) and loaded into the delivery capsule 212. In other examples, the prosthetic heart valve 900 can be radially compressed and loaded into delivery capsule 400. In certain examples, the recesses 404 of the delivery capsule 400 can be configured to receive the commissures 914 of the prosthetic heart valve 900 therein.

Additional details regarding prosthetic heart valves that can be used with the delivery capsules disclosed herein can be found, for example, in U.S. Pat. Nos. 8,652,202, 8,449,599, 9,393,110, 10,376,363, and 11,096,781, which are incorporated by reference herein.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for an expandable prosthetic heart valve, the delivery apparatus including a handle, and a shaft having a proximal end portion coupled to the handle and a distal end portion including a delivery capsule. The delivery capsule has an inner surface, an outer surface, and a plurality of elongate and axially extending inner and outer guide rails. The inner guide rails being spaced apart circumferentially around and extending radially inwardly from the inner surface and the outer guide rails being spaced apart circumferentially around and extending radially outwardly from the outer surface. The inner guide rails are configured to engage the prosthetic heart valve in a radially-compressed configuration and the outer guide rails are configured to contact an inner surface of an outer shaft.

Example 2. The delivery apparatus of example 1, wherein a total surface area of the inner surface is greater than a total surface area of the inner guide rails.

Example 3. The delivery apparatus of either example 1 or example 2, wherein a total surface area of the outer surface is greater than a total surface area of the outer guide rails.

Example 4. The delivery apparatus of any one of examples 1-3, wherein the inner guide rails are configured to slidably engage an outer surface of the prosthetic heart valve.

Example 5. The delivery apparatus of any one of examples 1-4, wherein the inner guide rails are configured to inhibit rotation of the prosthetic heart valve relative to the delivery capsule.

Example 6. The delivery apparatus of any one of examples 1-5, wherein one or more of the inner guide rails have a recess configured to retain one or more outwardly extending projections of the prosthetic heart valve while the prosthetic heart valve is in a radially-compressed configuration.

Example 7. The delivery apparatus of any one of examples 1-6, wherein each inner guide rail includes a recess configured to retain one or more outwardly extending projections of the prosthetic heart valve while the prosthetic heart valve is in a radially-compressed configuration.

Example 8. The delivery apparatus of either example 6 or example 7, wherein the inner guide rails comprise two parallel and opposing inner guide rails, and wherein the spacing between the opposing guide rails forms the recess configured to retain the outwardly extending projections of the prosthetic heart valve.

Example 9. The delivery apparatus of any one of examples 6-8, wherein the recess permits the outwardly extending projections of the prosthetic heart valve to move axially along the recess as the prosthetic heart valve slides axially along the shaft.

Example 10. The delivery apparatus of any one of examples 6-9, wherein the recess has a width greater than or equal to a width of the outwardly extending projections of the prosthetic heart valve such that the outwardly extending projections move axially along the recess as the prosthetic heart valve slides axially along the shaft.

Example 11. The delivery apparatus of any one of examples 1-10, wherein the inner surface forms an inner surface diameter and each inner guide rail has an outermost point relative to the inner surface from which the guide rail extends, and wherein the outermost points of the inner guide rails collectively form an inner rail diameter.

Example 12. The delivery apparatus of example 11, wherein the inner rail diameter is less than the inner surface diameter.

Example 13. The delivery apparatus of either example 11 or example 12, wherein the outer surface forms an outer surface diameter, and wherein the inner rail diameter is less than an outer surface diameter.

Example 14. The delivery apparatus of either example 12 or example 13, wherein the recess of each inner guide rail has an inner most point along an inner surface thereof, wherein the innermost points of the recesses collectively form a recess diameter, and wherein the recess diameter is greater than the inner rail diameter and less than the inner surface diameter.

Example 15. The delivery apparatus of any one of examples 1-14, wherein each outer guide rail has an outermost point relative to the outer surface from which the guide rail extends, and wherein the outermost points of the outer guide rails collectively form an outer rail diameter.

Example 16. The delivery apparatus of any one of examples 1-15, wherein the inner guide rails are configured to exert radial pressure against the prosthetic heart valve such that rotation of the prosthetic heart valve is inhibited.

Example 17. The delivery apparatus of any one of examples 1-16, wherein the handle is configured to apply rotation to the delivery capsule such that each of the inner guide rails and the prosthetic heart valve rotate relative to a native annulus.

Example 18. The delivery apparatus of any one of examples 1-17, wherein the delivery capsule is coupled to the shaft.

Example 19. The delivery apparatus of any one of examples 1-18, wherein the delivery capsule and the shaft are a unitary structure.

Example 20. The delivery apparatus of any one of examples 1-19, wherein one or more of the inner guide rails comprise a rounded cross-sectional profile taken in a plane perpendicular to a longitudinal axis of the shaft.

Example 21. The delivery apparatus of any one of examples 1-20, wherein one or more of the inner guide rails comprise a triangular cross-sectional profile taken in a plane perpendicular to a longitudinal axis of the shaft.

Example 22. The delivery apparatus of any one of examples 1-21, wherein one or more of the inner guide rails comprise a trapezoidal cross-sectional profile taken in a plane perpendicular to a longitudinal axis of the shaft.

Example 23. The delivery apparatus of any one of examples 1-22, wherein one or more of the inner guide rails comprise a rectangular cross-sectional profile taken in a plane perpendicular to a longitudinal axis of the shaft.

Example 24. The delivery apparatus of any one of examples 1-23, wherein each inner guide rail is radially aligned with a respective outer guide rail.

Example 25. The delivery apparatus of any one of examples 1-24, wherein each inner guide rail is radially offset from a pair of adjacent outer guide rails.

Example 26. The delivery apparatus of any one of examples 1-25, wherein each inner guide rail is equidistant from each adjacent inner guide rail.

Example 27. The delivery apparatus of any one of examples 1-26, wherein each outer guide rail is equidistant from each adjacent outer guide rail.

Example 28. A delivery apparatus for an expandable prosthetic heart valve, the delivery apparatus including a handle, and a first shaft and a second shaft extending over the first shaft, each shaft having a distal end portion and a proximal end portion coupled to the handle, the distal end portion of the second shaft having a delivery capsule including an inner surface, an outer surface, and a plurality of elongate positioning ribs extending axially along and circumferentially arranged around the inner surface and the outer surface. The positioning ribs of the inner surface are configured to capture the prosthetic heart valve in a radially-compressed configuration and the positioning ribs of the outer surface are configured to contact an outer shaft through which the delivery capsule can be inserted.

Example 29. The delivery apparatus of example 28, wherein the handle is configured to rotate the delivery capsule and the inner positioning ribs thereof, such that the prosthetic heart valve rotates relative to a native heart valve in a radially-compressed configuration.

Example 30. The delivery apparatus of either example 28 or example 29, wherein the outer shaft is an introducer configured to introduce the first shaft and the second shaft into a vasculature of a patient.

Example 31. The delivery apparatus of any one of examples 28-30, wherein the contact between the positioning ribs of the outer surface and an inner surface of the outer shaft reduces contact between the outer shaft and the outer surface of the second shaft.

Example 32. The delivery apparatus of any one of examples 28-31, wherein the positioning ribs of the inner surface comprise a groove configured to mate with an outwardly extending protrusion of the prosthetic heart valve.

Example 33. The delivery apparatus of example 32, wherein each groove has a width and a depth to receive and permit the outwardly extending protrusion of the prosthetic heart valve to move axially along the groove as the prosthetic heart valve slides longitudinally along the positioning ribs in the compressed configuration.

Example 34. The delivery apparatus of example 33, wherein the width and the depth of the grooves retain the outwardly extending protrusion within a space thereof such that the prosthetic heart valve is held rotationally stationary relative to the delivery capsule.

Example 35. The delivery apparatus of any one of examples 28-34, wherein the positioning ribs of the inner surface have a distal end and a proximal end, and wherein the positioning ribs comprise a tapered cross-sectional profile taken in a plane parallel to the central longitudinal axis of the delivery capsule at one of the distal end and the proximal end.

Example 36. The delivery apparatus of any one of examples 28-34, wherein the positioning ribs of the inner surface have a distal end and a proximal end, and wherein the positioning ribs comprise a tapered cross-sectional profile taken in a plane parallel to the central longitudinal axis of the delivery capsule surface at the distal end and at the proximal end.

Example 37. The delivery apparatus of any one of examples 28-36, wherein the positioning ribs of the outer surface have a distal end and a proximal end, and wherein the positioning ribs comprise a tapered cross-sectional profile taken in a plane parallel to the central longitudinal axis of the delivery capsule at one of the distal end and the proximal end.

Example 38. The delivery apparatus of any one of examples 28-36, wherein the positioning ribs of the outer surface have a distal end and a proximal end, and wherein the positioning comprise a tapered cross-sectional profile taken in a plane parallel to the central longitudinal axis of the delivery capsule at the distal end and at the proximal end.

Example 39. The delivery apparatus of any one of examples 28-35 or example 37, wherein the positioning ribs of the inner surface have a distal end and a proximal end, and wherein one of the distal end and the proximal end comprise a rectangular cross-sectional profile taken in a plane parallel to the central longitudinal axis of the delivery capsule.

Example 40. The delivery apparatus of any one of examples 28-36, wherein the positioning ribs of the outer surface have a distal end and a proximal end, and wherein one of the distal end and the proximal end comprise a rectangular cross-sectional profile taken in a plane parallel to the central longitudinal axis of the delivery capsule.

Example 41. The delivery apparatus of any one of examples 28-40, the delivery capsule having a distal end, a proximal end, and a length extending from the distal end to the proximal end, and wherein the positioning ribs of the inner surface have a length less than or equal to the length of the delivery capsule.

Example 42. The delivery apparatus of any one of examples 28-41, the delivery capsule having a distal end, a proximal end, and a length extending from the distal end to the proximal end, wherein the positioning ribs of the outer surface have a length less than or equal to the length of the delivery capsule.

Example 43. A method for delivering a prosthetic heart valve within a native annulus of a patient, the method includes advancing into a native vasculature of a patient a prosthetic heart valve mounted in a radially-compressed configuration around a distal end portion of a first shaft and engaged by a plurality of elongate guide rails spaced apart circumferentially within a delivery capsule of a second shaft extending over the distal end portion of the first shaft, wherein one or more outwardly extending protrusions of the prosthetic heart valve are received in a recess of the guide rails such that the prosthetic heart valve rotates with rotation of the delivery capsule, inserting the delivery capsule and the prosthetic heart valve into a native annulus of the patient, rotating the delivery capsule and prosthetic heart valve relative to the native vasculature and native annulus of the patient such that the prosthetic heart valve is oriented for implantation into the native annulus, retracting the second shaft such that the prosthetic heart valve and one or more outwardly extending protrusions move axially along the guide rails of the delivery capsule as the delivery capsule is withdrawn, and expanding the prosthetic heart valve from a radially-compressed configuration to a radially-expanded state within the native annulus.

Example 44. The method of example 43, wherein a tapered portion of the guide rails within the delivery capsule tapers from a longitudinal ridge thereof to an inner surface and a distal end section of the delivery capsule.

Example 45. The method of example 44, wherein as the second shaft is retracted the tapered portion directs the prosthetic heart valve away from the delivery capsule in a longitudinal direction.

Example 46. The method of example 44 or example 45, wherein the tapered portion of the guide rails within the delivery capsule tapers axially.

Example 47. The method of example 44 or example 45, wherein the tapered portion of the guide rails within the delivery capsule is curved in a circumferential direction.

Example 48. The method of any one of examples 43-47, further comprising introducing the first shaft, the second shaft, and a third shaft into the vasculature of the patient, wherein a plurality of elongate guide rails along an outer surface of the delivery capsule are in contact with and exert a radially outwardly force on an inner surface of the third shaft.

Example 49. The method of example 48, wherein a tapered portion of the guide rails along the outer surface tapers from a longitudinal ridge thereof to the outer surface and a proximal end section of the delivery capsule, and wherein as the first and second shafts are advanced into the native vasculature, the tapered portion of the guide rails along the outer surface of the delivery capsule directs the delivery capsule outward from the inner surface of the third shaft.

Example 50. The method of example 49, wherein the tapered portion of the guide rails along the outer surface tapers axially.

Example 51. The method of example 49, wherein the tapered portion of the guide rails along the outer surface is curved in a circumferential direction.

Example 52. The method of any one of examples 43-51, wherein each recess of the guide rails within the delivery capsule abuts with one or more outwardly extending protrusions received in the recess such that rotation of the delivery capsule causes the prosthetic heart valve to rotate with the delivery capsule.

Example 53. A method for positioning a prosthetic heart valve for implantation into an annulus of a patient, the method includes positioning a delivery capsule extending over a prosthetic heart valve in a radially-compressed configuration and mounted around a distal end portion of a shaft within an annulus of a patient, wherein the prosthetic heart valve is held rotationally stationary relative to the delivery capsule by a plurality of elongate and axially extending positioning ribs which mate with one or more outwardly extending protrusions of the prosthetic heart valve; and rotating and orienting via the delivery capsule and the positioning ribs thereof the prosthetic heart valve within the annulus of the patient such that one or more proximate native lumen are unobstructed upon implantation.

Example 54. An expandable prosthetic heart valve delivery assembly, the delivery assembly includes a delivery apparatus. The delivery apparatus includes a handle, a first shaft, and a second shaft extending over the first shaft, each shaft having a proximal end portion coupled to the handle and a distal end portion. The distal end portion of the second shaft includes a delivery capsule having an inner surface, an outer surface, and a plurality of elongate and axially extending inner and outer guide rails, each of the inner guide rails having a recess, and an expandable prosthetic heart valve mounted in a radially-compressed configuration around the distal end portion of the first shaft and retained within the delivery capsule of the second shaft. The recesses of the inner guide rails of delivery capsule mate with one or more outwardly extending protrusions of the prosthetic heart valve such that the prosthetic heart valve is rotationally stationary relative to the delivery capsule such that prosthetic heart valve rotates with rotation of the delivery capsule.

Example 55. The delivery assembly of example 54, the assembly further comprising an introducer having an inner surface and extending over the delivery capsule, wherein the outer guide rails of the delivery capsule contact the inner surface of the introducer such that contact between the outer surface of the delivery capsule and the inner surface of the introducer is reduced.

Example 56. The delivery assembly of either example 54 or example 55, wherein the inner guide rails are spaced apart circumferentially and equidistant around the inner surface.

Example 57. The delivery assembly of any one of examples 55-56, wherein the outer guide rails are spaced apart circumferentially and equidistant around the outer surface.

Example 58. A delivery capsule for a prosthetic heart valve delivery apparatus, the delivery capsule including a main body having an inner surface and an outer surface, a plurality of inner elongate ribs extending axially along the inner surface of the main body, and a plurality of outer elongate ribs extending axially along the outer surface of the main body. The inner ribs of the main body are configured to engage and hold a prosthetic heart valve rotationally stationary relative to the main body while the prosthetic heart valve is in a radially-compressed configuration.

In view of the many possible examples to which the principles of the disclosed invention may be applied, it should be recognized that the illustrated examples are only preferred examples and should not be taken as limiting the scope of the disclosure or the claimed subject matter. Rather, the scope of the claimed subject matter is defined by the following claims and their equivalents.

1. A delivery apparatus for an expandable prosthetic heart valve, the delivery apparatus comprising:
a handle; and
a shaft having a proximal end portion coupled to the handle and a distal end portion comprising a delivery capsule, the delivery capsule having an inner surface, an outer surface, and a plurality of elongate and axially extending inner guide rails and outer guide rails, the inner guide rails being spaced apart circumferentially around and extending radially inwardly from the inner surface, and the outer guide rails being spaced apart circumferentially around and extending radially outwardly from the outer surface,
wherein the inner guide rails are configured to engage the prosthetic heart valve in a radially-compressed configuration, and
wherein the outer guide rails are configured to contact an inner surface of an outer shaft.

2. The delivery apparatus of claim 1, wherein a total surface area of the inner surface is greater than a total surface area of the inner guide rails.

3. The delivery apparatus of claim 1, wherein a total surface area of the outer surface is greater than a total surface area of the outer guide rails.

4. The delivery apparatus of claim 1, wherein the inner guide rails are configured to slidably engage an outer surface of the prosthetic heart valve.

5. The delivery apparatus of claim 1, wherein the inner guide rails are configured to inhibit rotation of the prosthetic heart valve relative to the delivery capsule.

6. The delivery apparatus of claim 1, wherein one or more of the inner guide rails have a recess configured to retain one or more outwardly extending projections of the prosthetic heart valve while the prosthetic heart valve is in a radially-compressed configuration.

7. The delivery apparatus of claim 1, wherein each inner guide rail includes a recess configured to retain one or more outwardly extending projections of the prosthetic heart valve while the prosthetic heart valve is in a radially-compressed configuration.

8. The delivery apparatus of claim 7, wherein the inner guide rails comprise two parallel and opposing inner guide rails, and wherein spacing between the opposing inner guide rails forms the recess configured to retain the outwardly extending projections of the prosthetic heart valve.

9. The delivery apparatus of claim 7, wherein the recess permits the outwardly extending projections of the prosthetic heart valve to move axially along the recess as the prosthetic heart valve slides axially along the shaft.

10. The delivery apparatus of claim 7, wherein the recess has a width greater than or equal to a width of the outwardly extending projections of the prosthetic heart valve such that the outwardly extending projections move axially along the recess as the prosthetic heart valve slides axially along the shaft.

11. The delivery apparatus of claim 1, wherein the inner surface forms an inner surface diameter and each inner guide rail has an outermost point relative to the inner surface from which the inner guide rail extends, and wherein the outermost points of the inner guide rails collectively form an inner rail diameter.

12. The delivery apparatus of claim 11, wherein the inner rail diameter is less than the inner surface diameter.

13. The delivery apparatus of claim 11, wherein the outer surface forms an outer surface diameter, and wherein the inner rail diameter is less than an outer surface diameter.

14. The delivery apparatus of claim 13, wherein the recess of each inner guide rail has an innermost point along an inner surface thereof, wherein the innermost points of the recesses collectively form a recess diameter, and wherein the recess diameter is greater than the inner rail diameter and less than the inner surface diameter.

15. The delivery apparatus of claim 1, wherein each outer guide rail has an outermost point relative to the outer surface from which the outer guide rail extends, and wherein the outermost points of the outer guide rails collectively form an outer rail diameter.

16. A delivery apparatus for an expandable prosthetic heart valve, the delivery apparatus comprising:
a handle; and
a first shaft and a second shaft extending over the first shaft, each shaft having a distal end portion and a proximal end portion coupled to the handle, the distal end portion of the second shaft having a delivery capsule comprising an inner surface, an outer surface, and a plurality of elongate positioning ribs extending axially along and circumferentially arranged around the inner surface and the outer surface, wherein the positioning ribs of the inner surface are configured to capture the prosthetic heart valve in a radially-compressed configuration and the positioning ribs of the outer surface are configured to contact an outer shaft through which the delivery capsule can be inserted.

17. The delivery apparatus of claim 16, wherein the handle is configured to rotate the delivery capsule and the positioning ribs such that the prosthetic heart valve rotates relative to a native heart valve when the prosthetic heart valve is in the radially-compressed configuration.

18. A delivery apparatus for an expandable prosthetic heart valve, the delivery apparatus comprising:
a handle; and
a shaft having a proximal end portion coupled to the handle and a distal end portion comprising a delivery capsule, the delivery capsule having an inner surface, an outer surface, and a plurality of elongate and axially extending inner guide rails and outer guide rails,
wherein each of the inner guide rails is spaced apart circumferentially relative to an adjacent inner guide rail and extends radially inwardly from the inner surface of the delivery capsule, and
wherein each of the outer guide rails is spaced apart circumferentially relative to an adjacent outer guide member and extends radially outwardly from the outer surface of the delivery capsule.

19. The delivery apparatus of claim 18, wherein each of the inner guide rails is circumferentially aligned with one of the outer guide rails.

20. The delivery apparatus of claim 18, wherein each of the inner guide rails is circumferentially offset relative to the outer guide rails.
